(19) United States
(12) Patent Application Publication
(10) Pub. No.: US 2013/0053806 A1
(43) Pub. Date: Feb. 28, 2013

(54) PU FOAM COMPLEXED WITH NOVEFACE

(71) Applicants: Jean-Roger Guillo, Serezin de la tour (FR); Sophie Roland, Tignieu (FR); Vincent Chartron, Belleville (FR)

(72) Inventors: Jean-Roger Guillo, Serezin de la tour (FR); Sophie Roland, Tignieu (FR); Vincent Chartron, Belleville (FR)

(73) Assignee: ZODIAC AUTOMOTIVE DIVISION, Plaisir Cedex (FR)

(21) Appl. No.: 13/540,744

(22) Filed: Jul. 3, 2012

Related U.S. Application Data
(60) Provisional application No. 61/526,468, filed on Aug. 23, 2011.

(51) Int. Cl. A61F 13/537; B05D 7/00; A61F 13/534

(52) U.S. Cl. 604/369; 427/2.31

(57) ABSTRACT

A composite material for use in a medical device, such as a dressing, has an absorbent foam layer and a textile support layer having openings. A silicone-based gel impregnates the textile support without filling the openings in the support. The composite material is made by coating a textile structure with a silicone pre-polymer solution, depositing a polyurethane-based absorbent on one face of the coated textile structure and polymerizing the prepolymer solution.

### CROSS-REFERENCE TO RELATED APPLICATION

This application claims priority on U.S. Provisional Patent Appl. No. 61/526,468 filed on Aug. 23, 2011, the entire disclosure of which is incorporated herein by reference.

### BACKGROUND OF THE INVENTION

1. Field of the Invention

The invention concerns a composite material, in particular a material intended to be used in a medical device such as a dressing, as well as a method of manufacturing same. The invention also concerns a medical device comprising said composite material.

2. Description of the Related Art

Medical devices are known that are applied to a skin area to be protected having a lesion, for example a dressing. These devices isolate the skin area to be protected from any external contamination by surrounding fluids such as water, chemical substances such as soap or greases, and bacterial contamination agents or microorganisms. These devices also make it possible to discharge to the outside the excess water present inside said device and issuing from secretions on the skin area to be protected. Finally, they protect the covered skin area from any external mechanical attack.

Generally, such a device comprises an absorbent mass that absorbs the secretions from the skin area to be protected, such as the exudates from a wound, an adhesive layer that enables the device to adhere to the perilesional skin area and to be held around this area, and optionally a cover forming the external surface of said device.

Thus the absorbent mass absorbs the liquids secreted by the skin area.

Optionally, it may also contain active principles intended to treat the skin area.

A material for a medical device intended to be in contact with a wound is known through the document WO 87/05206, comprising an open structure, such as a knitted item, coated with a soft hydrophobic adhesive that adheres to the perilesional area but not to the wound.

The advantage of this soft adhesive is to make it possible for the removal of the composite material not to be painful, while promoting healing.

However, this material has a drawback of being difficult to process in practice. This is because, as it cannot absorb the exudates from the wound, it cannot be employed alone and must be used conjointly with an absorbent foam and a holding strip, the whole forming the medical device.

However, these various elements can only be assembled when the device is put in place since they do not have sufficient adhesion between them to allow cohesion over a long period, which requires frequent replacement of the medical device.

Moreover, a material for a medical device comprising an absorbent foam having openings that do not pass right through, coated with a hydrophobic adhesive gel that partially fills the openings without blocking them, is known through the document WO 97/42985.

However, this material has the drawback of being difficult to manufacture since producing holes in the absorbent foam that do not pass right through and then partially coating it so as not to block the bottom of the holes are tricky operations.

In addition, the interface with the skin area is provided solely by the soft hydrophobic adhesive gel, which is not very cohesive, which may lead to leaving residues on the skin when the medical device is removed.

The aim of the present invention is therefore to provide a composite material for a medical device intended to come into contact with a wound that is easy to manufacture and to integrate in a medical device.

### SUMMARY OF THE INVENTION

The invention concerns a composite material comprising a layer comprising an absorbent foam and a layer comprising a textile support having openings and a silicone-based gel, said silicone gel impregnating the textile support without filling the openings in said textile support.

The material according to the invention makes it possible to benefit from the gentle adhesion of the silicone gel and the absorbent capacity of the foam in a single unitary assembly having sufficient cohesion to be stored, manipulated, used and then removed without the different layers separating.

This is because the material according to the invention is a composite material, that is to say it is composed of several elements that nevertheless form a unit that keeps good cohesion.

The silicone gel impregnating the textile support has adhesive properties that hold together the coated textile support and the absorbent foam.

In addition, the silicone gel is a soft hydrophobic adhesive that adheres to the healthy skin but not to a wound (releasing aqueous exudates), so that it is not necessary to make provision for the gel to be present only at the areas of the material that are not intended to come into contact with a wound.

Finally, even if the silicone gel is hydrophobic, it can impregnate the entire surface of the textile structure since the latter comprises openings enabling the exudates of a wound to reach the absorbent foam.

Moreover, the use of a textile support makes the interface between the material of the invention and the skin much stronger than a silicone gel employed alone.

This is because the silicone gel is supported by the textile structure, which distributes the stresses of the entire coated surface at the time of removal.

Finally, the composite material according to the invention is easy to produce, as indicated below.

According to one embodiment of the invention, the absorbent foam is produced from a hydrophobic material permeable to fluids.

In a preferred embodiment of the invention, said absorbent foam is based on polyurethane.

For example, the absorbent foam is based on a hydrophobic cross-linked polyurethane not exhibiting any swelling during the absorption of fluids.

Alternatively, said absorbent mass is based on a non-woven fabric of fibres (for example based on cellulose or polypropylene).

The absorbent foam used typically has an absorption level of 10 and 30 g absorbed per g of foam.

It preferably has a thickness of between 1 and 10 mm.

Alternatively, the absorbent form has a thickness of less than 1 mm, in particular when the requirements in terms of absorption are small or the material is intended to be included in a complex comprising an additional absorbent foam.

The density of the foam is generally around 100 kg/m3.

The Lohman and Rauscher Superasorb X foam, the Corpura/AMS foam sold under the reference MCF03 or the MS 50PW foam from Filtrona can be cited by way of examples.

According to one embodiment of the invention, said foam contains therapeutic and/or prophylactic active principles that for example treat, accelerate healing or prevent infection of the skin area on which the material according to the invention will be placed.

For example, it will be possible to choose a foam capable of regulating the moisture level at the surface of the wound on which the composite material of the invention will be applied, thus promoting healing.

In general terms, the absorbent foam will be chosen according to the typology of the wound on which the composite material according to the invention is to be applied (slightly or greatly exudative wound).

Preferably, the textile structure used in the composite material is a knitted structure.

This is because knitting has the advantage of affording great flexibility, which enables the material to be comfortable and adapt to non-flat skin areas.

In addition, knitting technologies make it possible to obtain very varied types of stitch, with a very wide opening size range, which is not possible using a perforated hydrophobic gel alone.

The textile structure preferably comprises continuous filaments made from a synthetic material, for example based on polyester or polyamide.

The silicone gel forming part of the composition of the composite material according to the invention is chosen from medical grade silicones.

Examples of such silicones available commercially comprise the silicone sold by the company Dow Corning, under the reference MG 7-9800, or the silicone gels sold by the company Bluestar Silicone, under the reference HC2-2030.

The adhesion strength of the composite material on a skin area to be protected depends on the silicone gel chosen as well as its weight per square meter. A person skilled in the art will know how to adapt to the quantity of silicone gel to be used according to the nature thereof, so as to obtain an adhesion strength of the composite material on the skin of around 0.1 to 2 N/25 mm.

The silicone gel can impregnate the textile structure over the entire surface thereof; since the latter has openings, circulation of the liquid secreted by a wounded skin area will not be blocked.

In a variant, the silicone gel impregnates only part of the surface of the textile structure, for example the areas that are not intended to come into contact with a wound (perilesional areas).

The invention also concerns a medical device for protecting a skin area, such as a dressing, comprising the composite material described above, the layer comprising a textile support intended to be placed opposite the skin area to be protected.

The composite material according to the invention can be used directly as a medical device on a skin area to be protected.

In a variant, the medical device according to the invention comprises the composite material described above and other components.

Thus the medical device can be produced by assembling the composite material according to the invention with an external cover, for example an adhesive external cover that makes the device impervious to water and bacteria, thus allowing prolonged wearing thereof.

According to another example, the composite material according to the invention can be integrated in a medical device comprising an absorbent mass, for example an absorbent mass having particularly high absorption capacities.

The absorbent foam of the composite material will then be chosen so that it is hydrophobic but permeable to fluids and preferably non-swelling.

This is because medical devices of the type comprising an absorbent mass sandwiched between two layers of foam are known, one of the layers of foam constituting the interface with the skin area to be protected and the other constituting the external covering of the device.

However, it is difficult to make the interface adhere in contact with the skin area in these devices.

This is why it is particularly advantageous to replace the foam forming an interface with the skin area with the composite material according to the invention, which enables the medical device to adhere to the skin area but not to a wound, while remaining permeable to fluids.

In addition, the device that has been obtained has excellent airtightness.

The composite material and the absorbent mass can be assembled without chemical bonding, by wetting and mechanical attachment of the silicone gel in the pores of the absorbent mass, or on the other hand by chemical bonding, for example by coating an adhesion primer based on silicone and a reactive silane providing the chemical bonding between the functions of the silicone and that of the absorbent mass.

Since the composite material according to the invention has good thermal resistance, it and the foam layer serving as an external cover can be assembled by peripheral welding, for example by thermal welding or UHF welding.

The device thus obtained has excellent airtightness.

Because of this, the composite material according to the invention can be used in devices of the type described above and also provided with a hollow exit teat passing through the external cover of said device and enabling suction of the fluids absorbed by the absorbent mass and the application of a negative pressure to the wound by means of a device for putting under vacuum.

This type of device, known by the term NPW (negative pressure wounddressing) is known to promote healing, in particular in the case of wounds having high exudation.

In another case, the medical device may comprise the composite material described above as well as a holding strip or net.

Finally the invention concerns a method of manufacturing the composite material described above, said method comprising the steps consisting of;
coating a textile structure having openings with a silicone pre-polymer solution,
depositing a polyurethane based absorbent foam on one face of said coated textile structure, and
polymerising said pre-polymer solution.

The method according to the invention has the advantage of creating a complexed structure having particularly good cohesion.

This is because assembling the textile structure coated with a pre-polymer solution with the absorbent foam before proceeding with the polymerization enables the pre-polymer solution to be partially absorbed on the surface by the absorbent foam.

Thus, when the polymerization is carried out, this leads to the vulcanization of the pre-polymer solution and generates mechanical attachments between the absorbent foam and the textile structure.

When the absorbent foam is produced from polyurethane, a chemical attachment phenomenon is also created between the chains of the silicone and those of the polyurethane.

Because of these attachment phenomena, the adhesion between the textile structure and the absorbent mass is irreversible. The textile structure and the absorbent foam then form a unitary composite material that does not risk becoming disconnected.

In the context of the invention, it is also possible to use physical treatments, such as corona treatment, which significantly increase the attachment between the gel and the absorbent mass in the majority of cases.

The coating step can for example be carried out by coating with a scraper or cylinder or by pad finishing.

According to the viscosity of the pre-polymer solution employed, the latter will be absorbed by the absorbent foam to a greater or lesser extent, which makes it possible to vary the cohesion strength of the composite material according to the applications envisaged.

The quantity of silicone pre-polymer solution to be applied during the coating step depends on several factors and in particular the viscosity of the silicone. Generally the quantity of silicone applied during the coating step is from 100 to 1000 g/m2, depending on the properties of the silicone used and the adhesion strength between composite material and skin area sought (typically around 0.1 to 2 N).

The parameters for implementation of this coating step, such as the coating rate, are to be adapted according to the absorption capacity of the absorbent mass as well as the nature thereof, in order not to saturate it with silicone or to degrade it.

This is because the silicone pre-polymer solution must impregnate the absorbent foam solely on the surface to enable it to keep its capacity to absorb the exudates of a wound when the composite material is placed on the wounded skin area.

The coating may be continuous, over the entire textile structure, or only on part.

According to one embodiment of the invention, between the step of coating the textile structure and the step of depositing the absorbent foam, a step consisting of clearing the openings in the textile structure filled with the pre-polymer solution is carried out.

This embodiment is particularly suited when the pre-polymer solution used has high viscosity.

The polymerization step is performed according to conventional methods, in particular in a hot-air oven, fixing a temperature that the absorbent foam can withstand without degrading.

The invention will now be described in more detail with the help of the accompanying figures.

### BRIEF DESCRIPTION OF THE DRAWINGS

FIG. 1 is a view in section of a composite material according to the invention.

FIG. 2 is a plan view of the composite material of FIG. 1.

FIG. 3 is a view in section of a medical device according to the invention.

FIG. 4 is a view in section of an alternative embodiment of a medical device according to the invention.

FIG. 5 is a view in section of a variant of the medical device of FIG. 4.

### DETAILED DESCRIPTION OF THE PREFERRED EMBODIMENTS

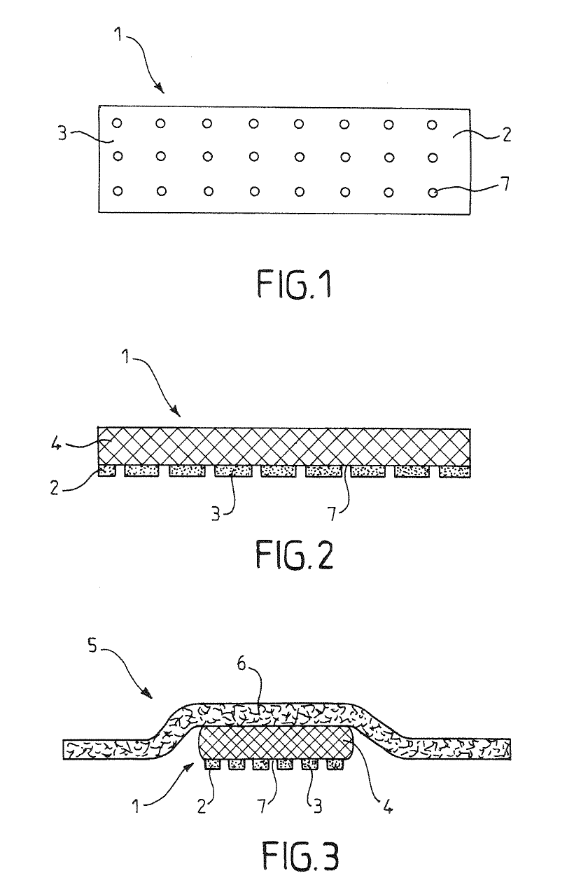

FIG. 1 and FIG. 2 show a composite material 1 according to the invention.

The composite material 1 is formed by a textile layer 2 having openings 7, coated with a silicone gel 3 and an absorbent foam 4.

Here the textile layer 2 is a layer made from a knitted item of continuous polyester filaments.

This knitted layer 2 is coated over its entire surface with a medical-grade silicone gel 3 that does not block the openings 7.

The silicone gel ensures cohesion with the absorbent foam 4, produced from polyurethane, with which it impregnates the surface.

FIG. 3 shows a medical device, here a dressing 5 comprising the composite material 1 described in FIGS. 1 and 2, as well as an external cover 7, the absorbent foam 4 being placed opposite the external cover 7.

This external cover 7 is here made from silicone.

Once the dressing 5 is in place on a skin area to be protected, the knitted layer 2 impregnated with silicone gel 3 comes into contact with said skin area to be protected, having for example a wound giving off exudates and a perilesional area.

Since the silicone gel 3 is hydrophobic, it does not adhere to the wound giving off exudates while the adhesion between the perilesional and the dressing 5 is gentle, so that removing said dressing 5 is not painful.

Because the silicone gel does not fill the openings 7, the exudates can pass through them and be absorbed by the absorbent foam 4.

Even once filled with exudates, the absorbent foam 4 remains secured to the knitted layer 2 coated with silicone gel 3, by virtue of the silicone gel that impregnates both the knitted layer 2 and the surface of the absorbent foam 4.

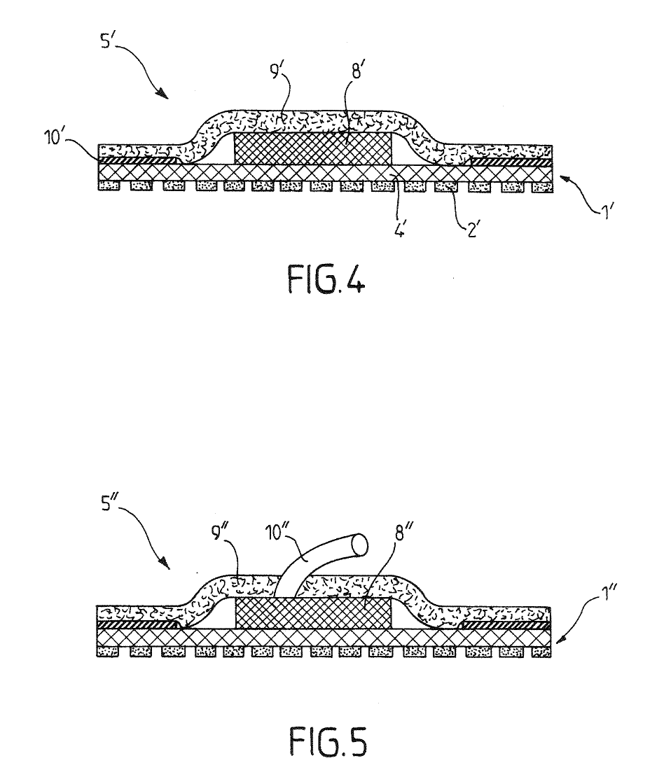

FIG. 4 shows another embodiment according to the invention, comprising the composite material 1′, intended to form the interface with a skin area to be protected, an absorbent mass 8′ and an external cover 9′ of the device.

The composite material 1′ comprises a knitted fabric 3 impregnated with silicone and an absorbent foam 4.

The external cover 9′ of the device 5′ is produced from an absorbent foam and fulfils the role of a membrane permeable to vapour. It is for example made from hydrophobic polyurethane.

The absorbent mass 8′ has very high absorption capacities and is for example produced from hydrophilic polyurethane.

The absorbent mass 8′ is sandwiched between the composite material 1′ and the absorbent foam 9′, which are connected to each other by peripheral welding (shown in FIG. 4 by the welding zones 10′).

Thus, once the medical device 5′ is arranged on a skin area to be protected, the secreted fluids pass through the composite material 1′ and can be absorbed in large quantities by the absorbent mass 8′.

FIG. 5 shows a variant 5″ of the medical device 5′, comprising, apart from a composite material 1″, an absorbent mass 8″ and an external cover 9″, a hollow teat 10″ for suction and putting under vacuum.

This teat is placed on the absorbent mass 8″ and passes through the external cover 9″.

This type of device is particularly advantageous for treating skin areas having particularly exudative wounds, the fitting of the teat making it possible, by means of a device for putting under vacuum, to create a negative pressure on the wound so that the liquids are discharged very effective.

What is claimed is:

1. A composite material comprising a layer comprising an absorbent foam and a layer comprising a textile support having openings and a silicone-based gel, said silicone gel impregnating the textile support without filling the openings in said textile support.

2. The composite material of claim 1, characterized in that said absorbent foam is based on polyurethane.

3. The composite material of claims 1, characterized in that said textile structure is a knitted structure.

4. The composite material of claim 1, characterized in that said textile support comprises continuous filaments made from a synthetic material.

5. The composite material of claim 1, characterized in that the polyurethane-based absorbent foam has a thickness of between 1 and 10 mm.

6. The composite material of claim 1, characterized in that the polyurethane-based absorbent foam contains therapeutic and/or prophylactic active principles.

7. A medical device for protecting a skin area, such as a dressing, comprising the composite material any one of claim 1, the layer comprising a textile support being intended to be placed opposite the skin area to be protected.

8. A method of manufacturing the composite material of claim 1, comprising the steps consisting of:
coating a textile structure having openings with a silicone pre-polymer solution,
depositing a polyurethane-based absorbent foam on one face of said coated textile structure, and
polymerizing said pre-polymer solution.

9. The method of claim 8, characterized in that the coating step is performed by scraper or cylinder or by pad finishing.

10. The method of claim 8, further comprising clearing the openings in the textile structure filled with the pre-polymer solution between the step of coating the textile structure and the step of depositing the absorbent foam.
